(19) United States
(12) Patent Application Publication
(10) Pub. No.: US 2023/0233465 A1
(43) Pub. Date: Jul. 27, 2023

(54) Formulation Comprising a Proteinaceous Microgel

(71) Applicant: University of Leeds, West Yorkshire (GB)

(72) Inventors: Anwesha Sarkar, West Yorkshire (GB); Efren Alberto Andablo Reyes, West Yorkshire (GB); Jing Hu, West Yorkshire (GB); Olivia Pabois, West Yorkshire (GB)

(21) Appl. No.: 17/999,209

(22) PCT Filed: May 19, 2021
(86) PCT No.: PCT/GB2021/051212

§ 371 Date: Nov. 17, 2022

(30) Foreign Application Priority Data
May 20, 2020 (GB) 2007546.1

(51) Int. Cl. A61K 9/107; A61K 38/40; A61K 47/36; A61K 38/16; B01J 13/00

(52) U.S. Cl. CPC A61K 9/1075; A61K 38/40; A61K 38/168; A61K 47/36; B01J 13/0065

(57) ABSTRACT

This invention relates to a formulation comprising a proteinaceous microgel and one or more biopolymeric nanofibrils. The invention also relates to methods for preparing such formulations. The invention also contemplates the uses of the formulations.

This invention relates to a formulation comprising a proteinaceous microgel and one or more biopolymeric nanofibrils. The invention also relates to methods for preparing such formulations. The invention also contemplates the uses of the formulations.

### BACKGROUND

Due to the growing cases of lubrication-failure related oral diseases, such as xerostomia (dry mouth), the development of new biocompatible lubricants with high performance under oral conditions has become an important research subject.

Xerostomia (the subjective sensation of dry mouth) is a common symptom with estimated prevalence of roughly 20% in the general population and up to 50% in the elderly [Furness S, Worthington HV, Bryan G, Birchenough S, McMillan R. Interventions for the management of dry mouth: topical therapies. Cochrane Database of Systematic Reviews. 2011; Hopcraft M, Tan C. Xerostomia: an update for clinicians. 2010;55:238-44].

The complaint of dry mouth can be related to objective symptoms of hyposalivation, such as: reduced salivary flow, change in the composition of saliva, or dry oral tissues, but it is also reported by people with normal salivary gland function [Villa A, Abati S. Risk factors and symptoms associated with xerostomia: a cross-sectional study. 2011;56:290-5]. There are several causes of xerostomia. A key clinical cause of dry mouth conditions is head and neck radiation therapy for cancers, which causes degeneration of salivary glands tissue, leading to reduction of saliva secretion depending on the radiation dose and treatment area [Lysik D, Niemirowicz-Laskowska K, Bucki R, Tokajuk G, Mystkowska J. Artificial Saliva: Challenges and Future Perspectives for the Treatment of Xerostomia. 2019;20:3199]. Other possible causes can be diseases including salivary gland diseases and disorders, chronic inflammatory autoimmune diseases like Sjögren’s syndrome, endocrine diseases like diabetes, neurologic diseases and disorders, psychogenic diseases and conditions like anxiety and nervousness, and infections like HIV/ AIDS [Furness S, Worthington HV, Bryan G, Birchenough S, McMillan R. Interventions for the management of dry mouth: topical therapies. Cochrane Database of Systematic Reviews. 2011; Lysik D, Niemirowicz-Laskowska K, Bucki R, Tokajuk G, Mystkowska J. Artificial Saliva: Challenges and Future Perspectives for the Treatment of Xerostomia. 2019;20:3199]. In addition, polypharmacy such as consuming multiple drugs at the same time including antihypertensives, opiates, antidepressants, antipsychotics, bronchodilators, proton pump inhibitors, antineoplastics, antihistamines, diuretics, and others can also induce dry mouth conditions [Porter SR, Scully C, Hegarty AM. An update of the etiology and management of xerostomia. Oral Surgery, Oral Medicine, Oral Pathology, Oral Radiology, and Endodontology. 2004;97:28-46; Thelin W, Brennan M, Lockhart P, Singh M, Fox P, Papas A, et al. The oral mucosa as a therapeutic target for xerostomia. 2008; 14:683-9].

Different therapies for xerostomia have been developed according to the diagnosis of the severity and causes [Narhi TO, Meurman JH, Ainamo A. Xerostomia and Hyposalivation. Drugs & Aging. 1999;15:103-16]. Typical treatment for xerostomia can involve the stimulation of the secretion of saliva, either pharmaceutically or by mechanical stimulation, and/or can involve symptomatic treatment like application of oral mucosal lubricants and/or salivary substitutes for the palliation of the symptoms [Łysik D, Niemirowicz-Laskowska K, Bucki R, Tokajuk G, Mystkowska J. Artificial Saliva: Challenges and Future Perspectives for the Treatment of Xerostomia. 2019;20:3199; Han P, Suarez-Durall P, Mulligan R. Dry mouth: A critical topic for older adult patients. Journal of Prosthodontic Research. 2015;59:6-19]. The symptomatic therapies generally aim at moistening the oral mucosa [Narhi TO, Meurman JH, Ainamo A. Xerostomia and hyposalivation: causes, consequences and treatment in the elderly. Drugs Aging. 1999;15:103-16]. Frequent fluid intake like water and glycerine or other biopolymers can be useful for periodic relief for dry mouth, but often the relief is short-lived. This is because most available therapies look at thickeners that just tend to increase the viscosity of water rather than focusing on lubrication properties, which are crucial aspects for salivary performance [Łysik D, Niemirowicz-Laskowska K, Bucki R, Tokajuk G, Mystkowska J. Artificial Saliva: Challenges and Future Perspectives for the Treatment of Xerostomia. 2019;20:3199]. Therefore, in the domain of salivary substitutes or oral moisturizers in the form of rinses, spray, gel, toothpastes or lozenges, there is a clear technology gap on systems that provide the necessary lubrication properties required for adequate treatment of xerostomia.

Recent studies have shown that microgels have potential to act as bio-lubricants [Andablo-Reyes E, Yerani D, Fu M, Liamas E, Connell S, Torres O, et al. Microgels as viscosity modifiers influence lubrication performance of continuum. Soft Matter. 2019;15:9614-24; Sarkar A, Kanti F, Gulotta A, Murray BS, Zhang S. Aqueous lubrication, structure and rheological properties of whey protein microgel particles. Langmuir. 2017;33:14699-708; Torres O, Andablo-Reyes E, Murray BS, Sarkar A. Emulsion microgel particles as high-performance bio-lubricants. ACS Applied Materials & Interfaces. 2018;10:26893-905] due to their capacity to reduce friction in soft contacts due to aqueous lubrication mechanism. However, the lubrication performance shown by microgels in relevant oral conditions is still poor in comparison to human saliva, which is an excellent bio-lubricant [Xu F, Liamas E, Bryant M, Adedeji AF, Andablo-Reyes E, Castronovo M, et al. A self-assembled binary protein model explains high-performance salivary lubrication from macro to nanoscale. Advanced Materials Interfaces. 2020;7:1901549].

Therefore, there is a need in the art for the provision of alternative or better treatments for xerostomia.

Swallowing disorder is one of the common results of dry mouth, which can significantly decrease the quality of life. Therefore, various compositions in the form of beverages or solid foods, such as chewing gum, candy and chocolate have been developed to facilitate mastication and deglutition of the food product [JP2018064512 “Solid food product easy in mastication and deglutition”; JP2016063832 “Packed beverage”]. For example, previous patents containing either salivary secretion promoting component or polysaccharide thickener have been reported. However, salivary secretion promoting components are effective only if there is remaining salivary function. Also, the efficiency of polysaccharide thickeners commonly used for such purpose have limited lubrication properties [Han, P., P. Suarez-Durall, and R. Mulligan, Dry mouth: a critical topic for older adult patients. J Prosthodont Res, 2015. 59(1): p. 6-19].

Therefore, there is a need in the art for the provision of alternative or better compositions that facilitate mastication and deglutition of food products.

### BRIEF SUMMARY OF THE DISCLOSURE

The present invention relates to aqueous formulations having bio-lubrication properties. Preferably, the formulations have improved bio-lubrication properties compared to commercially available bio-lubricants and human saliva.

The present invention relates to formulations having a low friction coefficient. Preferably, the formulations have a lower friction coefficient compared to commercially available bio-lubricants and human saliva.

The present invention relates to formulations having a low viscosity. Preferably, the formulations have lower viscosity compared to commercially available bio-lubricants.

In a first aspect of the present invention, there is provided a formulation comprising:
a proteinaceous or non-proteinaceous microgel; and
one or more biopolymeric nanofibrils;
wherein either one of: (i) the proteinaceous or non-proteinaceous microgel; and (ii) the one or more biopolymeric nanofibrils is positively charged, and the other is negatively charged; wherein the one or more biopolymeric nanofibrils are associated with an outer surface of the oppositely charged proteinaceous or non-proteinaceous microgel; and wherein the % outer surface coverage of the microgel by the nanofibrils is at least about 40%.

In a second aspect of the present invention, there is provided a formulation comprising:
a proteinaceous microgel; and
one or more biopolymeric nanofibrils;
wherein either one of: (i) the proteinaceous microgel; and (ii) the one or more biopolymeric nanofibrils is positively charged, and the other is negatively charged; wherein the one or more biopolymeric nanofibrils are associated with an outer surface of the oppositely charged proteinaceous microgel; and wherein the % outer surface coverage of the microgel by the nanofibrils is from about 50% to about 99%.

In a third aspect of the present invention, there is provided a method for preparing a formulation of the first aspect, the method comprising:
(a) dissolving a proteinaceous or non-proteinaceous material in a buffer solution and heating the resulting solution to form a heat-set gel;
(b) mixing the heat-set gel with the buffer solution and homogenising to form a proteinaceous or non-proteinaceous microgel;
(c) adding the proteinaceous or non-proteinaceous microgel to a solution of one or more biopolymeric nanofibrils to form the formulation,
wherein either one of: (i) the proteinaceous or non-proteinaceous microgel; and (ii) the one or more biopolymeric nanofibrils is positively charged, and the other is negatively charged; wherein the resulting formulation has the one or more biopolymeric nanofibrils associated with an outer surface of the oppositely charged proteinaceous or non-proteinaceous microgel; and wherein the amount of microgel that is added to nanofibrils is selected such that the % outer surface coverage of the microgel by the nanofibrils is at least about 40%.

In a fourth aspect of the present invention, there is provided a method for preparing a formulation of the second aspect, the method comprising:
(a) dissolving a proteinaceous material in a buffer solution and heating the resulting solution to form a proteinaceous microgel or a heat-set gel;
(b) when step (a) results in a heat-set gel, mixing the heat-set gel with the buffer solution and homogenising to form a proteinaceous microgel;
(c) adding the proteinaceous microgel of step (a) or step (b) to a solution of one or more biopolymeric nanofibrils to form the formulation,
wherein either one of: (i) the proteinaceous microgel; and (ii) the one or more biopolymeric nanofibrils is positively charged, and the other is negatively charged; wherein the resulting formulation has the one or more biopolymeric nanofibrils associated with an outer surface of the oppositely charged proteinaceous microgel; and wherein the amount of microgel that is added to nanofibrils is selected such that the % outer surface coverage of the microgel by the nanofibrils is from about 50% to about 99%.

In a fifth aspect of the present invention, there is provided a formulation obtainable or obtained by the method of the third or fourth aspect.

In a sixth aspect of the present invention, there is provided a formulation of the first or second aspect for use as a medicament.

In a seventh aspect of the present invention, there is provided a use of a formulation of the first or second aspect as a lubricant food additive, i.e. for fat replacement purposes.

In an eighth aspect of the present invention, there is provided a formulation of the first or second aspect for use in the treatment of a disease or condition selected from or associated with: dry mouth, salivary gland diseases and disorders, chronic inflammatory autoimmune diseases, Sjogren’s syndrome, xerostomia, endocrine diseases, dysphagia, diabetes, neurologic diseases and disorders, psychogenic diseases, anxiety, nervousness, aging, HIV/AIDS and polypharmacy.

In an ninth aspect of the present invention, there is provided a formulation of the first or second aspect for use in the treatment of a disease or condition selected from or associated with: dry mouth, salivary gland diseases and disorders, chronic inflammatory autoimmune diseases, Sjögren’s syndrome, xerostomia, endocrine diseases, diabetes, neurologic diseases and disorders, psychogenic diseases, anxiety, nervousness, aging, HIV/AIDS and polypharmacy.

In a tenth aspect of the present invention, there is provided a method for the treatment of a disease or condition selected from or associated with: dry mouth, salivary gland diseases and disorders, chronic inflammatory autoimmune diseases, Sjögren’s syndrome, xerostomia, endocrine diseases, dysphagia, diabetes, neurologic diseases and disorders, psychogenic diseases, anxiety, nervousness, aging, HIV/AIDS and polypharmacy, wherein the method comprises administering a formulation of the first or second aspect to a patient in need thereof.

In an eleventh aspect of the present invention, there is provided a method for the treatment of a disease or condition selected from or associated with: dry mouth, salivary gland diseases and disorders, chronic inflammatory autoimmune diseases, Sjögren’s syndrome, xerostomia, endocrine diseases, diabetes, neurologic diseases and disorders, psychogenic diseases, anxiety, nervousness, aging, HIV/AIDS and polypharmacy, wherein the method comprises administering a formulation of the first or second aspect to a patient in need thereof.

### BRIEF DESCRIPTION OF THE DRAWINGS

Embodiments of the invention are further described hereinafter with reference to the accompanying drawings, in which:

FIG. 1 shows TEM images of examples of the formulation of the present invention made following Example 1. (a) TEM image of lactoferrin microgel (LFM) with scale bar of 500 nm. (b) TEM image of κ-carrageenan nanofibrils (KCnF) with scale bar of 500 nm. (c) TEM image of LFM partially covered by KCnF with scale bar of 500 nm, and (d1)(d2) with scale bar of 200 nm.

FIG. 2 shows examples of the formulation of the present invention made following Example 1. (a) Schematic diagram of LFM coated with KCnF. (b) Photograph of a formulation of the present invention shown under uniaxial extensional flow exerted manually between fingertips (thumb and forefinger), such behaviour is also shown by real human saliva. (c) Schematic of the structure created by the formulation containing a plurality of LFM microgel particles connected by interaction of adjacent KCnF nanofibrils.

FIG. 3 shows the ζ-potential of the formulation as a function of different weight ratios of KCnF:LFM (from 0.01:1 to 3:1 w/w) made following Example 1. As the relative concentration of KCnF increases, a steep inversion in the sign of ζ-potential is observed.

FIG. 4 shows the shear viscosity at the orally relevant shear rate of LFM, KCnF, and exemplary formulations of the invention made following Example 1 (comprising KCnF and LFM in a ratio of 0.01:1 to 3:1 w/w).

FIG. 5 shows the shear viscosity at the orally relevant shear rate of exemplary formulations of the invention made following Example 1 (comprising KCnF and LFM in a ratio of 0.01:1 and 0.60:1 w/w), honey, human saliva, and various commercially available saliva replacement gel and spray products.

FIG. 6a and FIG. 6b shows the friction coefficients as a function of speed obtained for exemplary formulations of the present invention made following Example 1 (comprising KCnF and LFM in a ratio of 0.01:1 to 3:1 w/w), and compares these values with those obtained for LFM, KCnF, human saliva and buffer. The data provided in FIG. 6b is the same data as is that in FIG. 6a. The Figures differ in that the y-axis has been altered from 0.001-10 (in FIG. 6a) to 0.002-2 (in FIG. 6b).

FIG. 7 shows the friction coefficients as a function of speed obtained for one exemplary formulation of the present invention made following Example 1 (comprising KCnF and LFM in a ratio of 0.60:1 w/w), and compares these values with those obtained for human saliva and various commercially available saliva replacement gel and spray products.

FIG. 8 shows the ζ-potential of the formulation as a function of different weight ratios of KCnF:LFM (from 0.01:1 to 2:1 w/w) made following Example 2, and compares these values with those obtained for KCnF and LFM. As the relative concentration of KCnF increases, a steep inversion in the sign of ζ-potential is observed.

FIG. 9 shows the shear viscosity at the orally relevant shear rate of LFM, KCnF, and exemplary formulations of the invention made following Example 2 (comprising KCnF and LFM in a ratio of 0.01:1 to 2:1 w/w).

FIG. 10 shows the friction coefficients as a function of speed obtained for exemplary formulations of the present invention made following Example 2 (comprising KCnF and LFM in a ratio of 0.01:1 to 2:1 w/w), and compares these values with those obtained for LFM, KCnF, human saliva and buffer.

FIG. 11 shows the friction coefficients as a function of speed obtained for exemplary formulations of the present invention made following Example 2 (comprising KCnF and LFM in a ratio of 0.60:1 w/w) at 0 month, 1 month and 2 months storage.

FIG. 12 shows the ζ-potential of the formulation as a function of different weight ratios of AnF:LFM (from 0.01:1 to 1:1 w/w) made following Example 3, and compares these values with those obtained for AnF and LFM.

FIG. 13 shows the shear viscosity at the orally relevant shear rate of LFM, AnF, and exemplary formulations of the invention made following Example 3 (comprising AnF and LFM in a ratio of 0.01:1 to 1:1 w/w).

FIG. 14 shows the friction coefficients as a function of speed obtained for exemplary formulations of the present invention made following Example 3 (comprising AnF and LFM in a ratio of 0.01:1 to 1:1 w/w), and compares these values with those obtained for LFM, AnF, human saliva and buffer.

FIG. 15 shows the ζ-potential of the formulation as a function of different weight ratios of KCnF: PoPM (from 0.01:1 to 2:1 w/w) made following Example 4, and compares these values with those obtained for KCnF and PoPM.

FIG. 16 shows the shear viscosity at the orally relevant shear rate of PoPM, KCnF, and exemplary formulations of the invention made following Example 4 (comprising KCnF and PoPM in a ratio of 0.01:1 to 2:1 w/w).

FIG. 17 shows the friction coefficients as a function of speed obtained for exemplary formulations of the present invention made following Example 4 (comprising KCnF and PoPM in a ratio of 0.01:1 to 2:1 w/w), and compares these values with those obtained for PoPM, KCnF, human saliva and buffer.

FIG. 18 shows the ζ-potential of the formulation as a function of different weight ratios of XGnF: PoPM (from 0.01:1 to 2:1 w/w) made following Example 5, and compares these values with those obtained for XGnF and PoPM.

FIG. 19 shows the shear viscosity at the orally relevant shear rate of PoPM, XGnF, and exemplary formulations of the invention made following Example 5 (comprising XGnF and PoPM in a ratio of 0.01:1 to 2:1 w/w).

FIG. 20 shows the friction coefficients as a function of speed obtained for exemplary formulations of the present invention made following Example 5 (comprising XGnF and PoPM in a ratio of 0.01:1 to 2:1 w/w), and compares these values with those obtained for PoPM, XGnF, human saliva and buffer.

### DETAILED DESCRIPTION

The abbreviations used herein have their conventional meaning within the chemical and biological arts.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader’s attention is directed to all papers and documents which are filed concurrently with or previous to this specification in connection with this application and which are open to public inspection with this specification, and the contents of all such papers and documents are incorporated herein by reference.

For the avoidance of doubt, it is hereby stated that the information disclosed earlier in this specification under the heading “Background” is relevant to the invention and is to be read as part of the disclosure of the invention.

### Definitions

The term ‘microgel’ includes a particle of gel of any shape with an equivalent diameter of approximately 0.05 to 100 µm.

The term ‘nanofibril’ includes a tubular-shaped structure of any polymer with an equivalent diameter of approximately 1 to 100 nm.

The term ‘colloidosome’ includes a core-shell system having a colloidal core and a shell composed of colloidal particles or fibrils.

### Formulations

In an embodiment, the proteinaceous or non-proteinaceous microgel is positively charged and the one or more biopolymeric nanofibrils are negatively charged. In an alternative embodiment, the proteinaceous or non-proteinaceous microgel is negatively charged and the one or more biopolymeric nanofibrils are positively charged. In a preferred embodiment, the proteinaceous or non-proteinaceous microgel is positively charged and the one or more biopolymeric nanofibrils are negatively charged.

In embodiments, the one or more biopolymeric nanofibrils are associated with an outer surface of the proteinaceous or non-proteinaceous microgel by an electrostatic interaction. The association of the biopolymeric nanofibrils with an outer surface of the proteinaceous or non-proteinaceous microgel may be regarded as a form of ‘coating’ of the biopolymeric nanofibrils onto the outer surface of the proteinaceous or non-proteinaceous microgel. The association (or coating) of the biopolymeric nanofibrils with an outer surface of the proteinaceous or non-proteinaceous microgel results in an arrangement whereby the microgel is surrounded by a permeable mesh of biopolymeric nanofibrils of different local concentrations on the outer surface of the proteinaceous or non-proteinaceous microgel. In an embodiment, the association between the biopolymeric nanofibrils and the outer surface of the proteinaceous or non-proteinaceous microgel is a direct association, i.e., the biopolymeric nanofibrils and the outer surface of the proteinaceous or non-proteinaceous microgel are associated with one another in the absence of an intermediate component. In an embodiment, the formulation of the present invention consists of only two oppositely charged components (i.e., the proteinaceous or non-proteinaceous microgel and the one or more biopolymeric nanofibrils). As explained above, the two components of the formulation of the invention interact with each other via direct, electrostatic interactions, thus allowing the microgel particle to be coated with oppositely-charged biopolymeric nanofibrils.

In embodiments, the one or more biopolymeric nanofibrils associated with the outer surface of the proteinaceous or non-proteinaceous microgel result in an outer surface that has an overall negative charge. In alternative embodiments, the one or more biopolymeric nanofibrils associated with the outer surface of the proteinaceous or non-proteinaceous microgel result in an outer surface that has an overall positive charge.

In embodiments, the proteinaceous or non-proteinaceous microgel is selected from the group consisting of: lactoferrin, lysozyme, gelatin, milk protein, bovine serum albumin, whey protein, casein, caseinate, egg protein, albumin, gluten, gelatin Type B, pea protein, rice protein, legumin, corn protein, peanut protein, chitosan, chitin and potato protein.

In embodiments, the proteinaceous or non-proteinaceous microgel is selected from the group consisting of: lactoferrin, lysozyme, gelatin, milk protein, bovine serum albumin, whey protein, caseinate, egg protein, albumin, gluten, gelatin Type B, pea protein, rice protein, legumin, corn protein, peanut protein, chitosan and chitin.

In embodiments, the microgel is a proteinaceous microgel. This embodiment is the preferred embodiment of the present invention. In embodiments, the proteinaceous microgel is selected from the group consisting of: lactoferrin, lysozyme, gelatin, milk protein, bovine serum albumin, whey protein, casein, caseinate, egg protein, albumin, gluten, gelatin Type B, pea protein, rice protein, legumin, corn protein, peanut protein and potato protein. In embodiments, the proteinaceous microgel is selected from the group consisting of: lactoferrin, lysozyme, gelatin, milk protein, bovine serum albumin, whey protein, caseinate, egg protein, albumin, gluten, gelatin Type B, pea protein, rice protein, legumin, corn protein and peanut protein. In a preferred embodiment, the microgel is lactoferrin. In a preferred embodiment, the microgel is potato protein.

In embodiments, the microgel is a non-proteinaceous microgel. In embodiments, the non-proteinaceous microgel is selected from the group consisting of: chitosan and chitin.

In embodiments, the microgel is charged at a pH of from about 3.0 to about 7.0. In such embodiments, the microgel is selected from the group consisting of: lactoferrin, lysozyme, gelatin Type B, chitosan and chitin. Preferably, the microgel is charged at a pH of about 7.0.

In embodiments, the microgel is charged at a pH of from about 3.0 to about 4.0. In such embodiments, the proteinaceous is selected from the group consisting of: gelatin, milk protein, bovine serum albumin, whey protein, casein, caseinate, egg protein, albumin, gluten, pea protein, potato protein, rice protein, legumin, corn protein and peanut protein.

In embodiments, the microgel is charged at a pH of from about 3.0 to about 4.0. In such embodiments, the proteinaceous or non-proteinaceous microgel is selected from the group consisting of: gelatin, milk protein, bovine serum albumin, whey protein, caseinate, egg protein, albumin, gluten, pea protein, rice protein, legumin, corn protein, and peanut protein.

In embodiments, the microgel is no more than 500 nm in diameter. In embodiments, the microgel has a diameter of from about 50 nm to about 500 nm. In embodiments, the microgel has a diameter of from about 60 nm to about 500 nm. In embodiments, the microgel has a diameter of about 70 nm to about 500 nm. In embodiments, the microgel has a diameter of about 80 nm to about 500 nm. In embodiments, the microgel has a diameter of about 90 nm to about 500 nm. In embodiments, the microgel has a diameter of about 100 nm to about 500 nm.

In embodiments, the microgel is no more than 400 nm in diameter. In embodiments, the microgel has a diameter of from about 50 nm to about 400 nm. In embodiments, the microgel has a diameter of from about 60 nm to about 400 nm. In embodiments, the microgel has a diameter of about 70 nm to about 400 nm. In embodiments, the microgel has a diameter of about 80 nm to about 400 nm. In embodiments, the microgel has a diameter of about 90 nm to about 400 nm. In embodiments, the microgel has a diameter of about 100 nm to about 400 nm.

In embodiments, the microgel is no more than 300 nm in diameter. In embodiments, the microgel has a diameter of from about 50 nm to about 300 nm. In embodiments, the microgel has a diameter of from about 60 nm to about 300 nm. In embodiments, the microgel has a diameter of about 70 nm to about 300 nm. In embodiments, the microgel has a diameter of about 80 nm to about 300 nm. In embodiments, the microgel has a diameter of about 90 nm to about 300 nm. In embodiments, the microgel has a diameter of about 100 nm to about 300 nm.

In embodiments, the microgel has a diameter of no more than 200 nm. In embodiments, the microgel has a diameter of from about 50 nm to about 200 nm. In embodiments, the microgel has a diameter of from about 60 nm to about 200 nm. In embodiments, the microgel has a diameter of about 70 nm to about 200 nm. In a preferred embodiment, the microgel has a diameter of about 80 nm to about 200 nm. In embodiments, the microgel has a diameter of about 90 nm to about 200 nm. In embodiments, the microgel has a diameter of about 100 nm to about 200 nm.

In embodiments, the formulation of the first aspect may comprise a plurality of biopolymeric nanofibrils. Alternatively, the formulation of the first aspect may comprise one biopolymeric nanofibril.

The one or more biopolymeric nanofibrils may be polysaccharide-based nanofibrils. In embodiments, the one or more nanofibrils are selected from the group consisting of: κ-carrageenan, I-carrageenan, λ-carrageenan, agar, agarose, alginate, pectin, dextran sulphate, cellulose, xanthan gum, gellan gum, and any negatively charged polysaccharide. In a preferred embodiment, the one or more biopolymeric nanofibrils are κ-carrageenan nanofibrils. In a preferred embodiment, the one or more biopolymeric nanofibrils are made by addition of agar. In a preferred embodiment, the one or more biopolymeric nanofibrils are made by addition of xanthan gum.

In embodiments, the one or more biopolymeric nanofibrils are no more than 50 nm in diameter.

In embodiments, the one or more biopolymeric nanofibrils have a diameter of from about 1 nm to about 50 nm. In embodiments, the one or more biopolymeric nanofibrils have a diameter of from about 5 nm to about 50 nm. In embodiments, the one or more biopolymeric nanofibrils have a diameter of from about 10 nm to about 50 nm.

In embodiments, the one or more biopolymeric nanofibrils have a diameter of from about 1 nm to about 40 nm. In embodiments, the one or more biopolymeric nanofibrils have a diameter of from about 1 nm to about 30 nm. In embodiments, the one or more biopolymeric nanofibrils have a diameter of from about 1 nm to about 20 nm.

In embodiments, the one or more biopolymeric nanofibrils have a diameter of from about 5 nm to about 40 nm. In embodiments, the one or more biopolymeric nanofibrils have a diameter of from about 5 nm to about 30 nm. In embodiments, the one or more biopolymeric nanofibrils have a diameter of from about 5 nm to about 20 nm.

In embodiments, the one or more biopolymeric nanofibrils have a diameter of from about 10 nm to about 40 nm. In embodiments, the one or more biopolymeric nanofibrils have a diameter of from about 10 nm to about 30 nm. In embodiments, the one or more biopolymeric nanofibrils have a diameter of from about 10 nm to about 20 nm.

Preferably, the one or more biopolymeric nanofibrils have a diameter of from about 5 nm to about 20 nm.

In embodiments, the one or more biopolymeric nanofibrils are no more than 500 nm in length.

In embodiments, the one or more biopolymeric nanofibrils have a length of from about 50 nm to about 500 nm. In embodiments, the one or more biopolymeric nanofibrils have a length of from about 75 nm to about 500 nm. In embodiments, the one or more biopolymeric nanofibrils have a length of from about 100 nm to about 500 nm.

In embodiments, the one or more biopolymeric nanofibrils have a length of from about 50 nm to about 400 nm. In embodiments, the one or more biopolymeric nanofibrils have a length of from about 75 nm to about 400 nm. In embodiments, the one or more biopolymeric nanofibrils have a length of from about 100 nm to about 400 nm.

In embodiments, the one or more biopolymeric nanofibrils have a length of from about 50 nm to about 300 nm. In embodiments, the one or more biopolymeric nanofibrils have a length of from about 75 nm to about 300 nm. In embodiments, the one or more biopolymeric nanofibrils have a length of from about 100 nm to about 300 nm.

In embodiments, the one or more biopolymeric nanofibrils have a length of from about 50 nm to about 475 nm. In embodiments, the one or more biopolymeric nanofibrils have a length of from about 50 nm to about 450 nm. In embodiments, the one or more biopolymeric nanofibrils have a length of from about 50 nm to about 425 nm. In embodiments, the one or more biopolymeric nanofibrils have a length of from about 50 nm to about 400 nm. In embodiments, the one or more biopolymeric nanofibrils have a length of from about 50 nm to about 375 nm. In embodiments, the one or more biopolymeric nanofibrils have a length of from about 50 nm to about 350 nm. In embodiments, the one or more biopolymeric nanofibrils have a length of from about 50 nm to about 325 nm. In embodiments, the one or more biopolymeric nanofibrils have a length of from about 50 nm to about 300 nm.

In embodiments, the one or more biopolymeric nanofibrils have a length of from about 75 nm to about 475 nm. In embodiments, the one or more biopolymeric nanofibrils have a length of from about 75 nm to about 450 nm. In embodiments, the one or more biopolymeric nanofibrils have a length of from about 75 nm to about 425 nm. In embodiments, the one or more biopolymeric nanofibrils have a length of from about 75 nm to about 400 nm. In embodiments, the one or more biopolymeric nanofibrils have a length of from about 75 nm to about 375 nm. In embodiments, the one or more biopolymeric nanofibrils have a length of from about 75 nm to about 350 nm. In embodiments, the one or more biopolymeric nanofibrils have a length of from about 75 nm to about 325 nm. In embodiments, the one or more biopolymeric nanofibrils have a length of from about 75 nm to about 300 nm.

In embodiments, the one or more biopolymeric nanofibrils have a length of from about 100 nm to about 475 nm. In embodiments, the one or more biopolymeric nanofibrils have a length of from about 100 nm to about 450 nm. In embodiments, the one or more biopolymeric nanofibrils have a length of from about 100 nm to about 425 nm. In embodiments, the one or more biopolymeric nanofibrils have a length of from about 100 nm to about 400 nm. In embodiments, the one or more biopolymeric nanofibrils have a length of from about 100 nm to about 375 nm. In embodiments, the one or more biopolymeric nanofibrils have a length of from about 100 nm to about 350 nm. In embodiments, the one or more biopolymeric nanofibrils have a length of from about 100 nm to about 325 nm. In embodiments, the one or more biopolymeric nanofibrils have a length of from about 100 nm to about 300 nm.

In a preferred embodiment, the one or more biopolymeric nanofibrils have a length of from about 100 nm to about 300 nm.

The % outer surface coverage of the microgel by the nanofibrils is at least about 40% (e.g., at least about 41%, at least about 42%, at least about 43% or at least about 44%). In embodiments, the % outer surface coverage is at least about 45% (e.g., at least about 46%, at least about 47%, at least about 48% or at least about 49%). In embodiments, the % outer surface coverage is at least about 50% (e.g., at least about 51%, at least about 52%, at least about 53% or at least about 54%). In embodiments, the % outer surface coverage is at least about 55% (e.g., at least about 56%, at least about 57%, at least about 58% or at least about 59%). In embodiments, the % outer surface coverage is at least about 60% (e.g., at least about 61%, at least about 62%, at least about 63% or at least about 64%). In embodiments, the % outer surface coverage is at least about 65% (e.g., at least about 66%, at least about 67%, at least about 68% or at least about 69%). In embodiments, the % outer surface coverage is at least about 70% (e.g., at least about 71%, at least about 72%, at least about 73% or at least about 74%). In embodiments, the % outer surface coverage is at least about 75% (e.g., at least about 76%, at least about 77%, at least about 78% or at least about 79%). In embodiments, the % outer surface coverage is at least about 80% (e.g., at least about 81%, at least about 82%, at least about 83% or at least about 84%). In embodiments, the % outer surface coverage is at least about 85% (e.g., at least about 86%, at least about 87%, at least about 88% or at least about 89%). In embodiments, the % outer surface coverage is at least about 90%.

In embodiments, the % outer surface coverage is from about 40% to about 99%. In embodiments, the % outer surface coverage is from about 40% to about 95%. In embodiments, the % outer surface coverage is from about 40% to about 90%. In embodiments, the % outer surface coverage is from about 40% to about 85%. In embodiments, the % outer surface coverage is from about 40% to about 80%. In embodiments, the % outer surface coverage is from about 40% to about 75%. In embodiments, the % outer surface coverage is from about 40% to about 70%. In embodiments, the % outer surface coverage is from about 40% to about 60%.

In embodiments, the % outer surface coverage is from about 45% to about 99%. In embodiments, the % outer surface coverage is from about 45% to about 95%. In embodiments, the % outer surface coverage is from about 45% to about 90%. In embodiments, the % outer surface coverage is from about 45% to about 85%. In embodiments, the % outer surface coverage is from about 45% to about 80%. In embodiments, the % outer surface coverage is from about 45% to about 75%. In embodiments, the % outer surface coverage is from about 45% to about 70%. In embodiments, the % outer surface coverage is from about 45% to about 60%.

In embodiments, the % outer surface coverage is from about 50% to about 99%. In embodiments, the % outer surface coverage is from about 50% to about 95%. In embodiments, the % outer surface coverage is from about 50% to about 90%. In embodiments, the % outer surface coverage is from about 50% to about 85%. In embodiments, the % outer surface coverage is from about 50% to about 80%. In embodiments, the % outer surface coverage is from about 50% to about 75%. In embodiments, the % outer surface coverage is from about 50% to about 70%. In embodiments, the % outer surface coverage is from about 50% to about 60%.

In embodiments, the % outer surface coverage is from about 55% to about 99%. In embodiments, the % outer surface coverage is from about 55% to about 95%. In embodiments, the % outer surface coverage is from about 55% to about 90%. In embodiments, the % outer surface coverage is from about 55% to about 85%. In embodiments, the % outer surface coverage is from about 55% to about 80%. In embodiments, the % outer surface coverage is from about 55% to about 75%. In embodiments, the % outer surface coverage is from about 55% to about 70%. In embodiments, the % outer surface coverage is from about 55% to about 60%.

In embodiments, the % outer surface coverage is from about 60% to about 99%. In embodiments, the % outer surface coverage is from about 60% to about 95%. In embodiments, the % outer surface coverage is from about 60% to about 90%. In embodiments, the % outer surface coverage is from about 60% to about 85%. In embodiments, the % outer surface coverage is from about 60% to about 80%. In embodiments, the % outer surface coverage is from about 60% to about 75%. In embodiments, the % outer surface coverage is from about 60% to about 70%.

In embodiments, the % outer surface coverage is from about 65% to about 99%. In embodiments, the % outer surface coverage is from about 65% to about 95%. In embodiments, the % outer surface coverage is from about 65% to about 90%. In embodiments, the % outer surface coverage is from about 65% to about 85%. In embodiments, the % outer surface coverage is from about 65% to about 80%. In embodiments, the % outer surface coverage is from about 65% to about 75%. In embodiments, the % outer surface coverage is from about 65% to about 70%.

In embodiments, the % outer surface coverage is from about 70% to about 99%. In embodiments, the % outer surface coverage is from about 70% to about 95%. In embodiments, the % outer surface coverage is from about 70% to about 90%. In embodiments, the % outer surface coverage is from about 70% to about 85%. In embodiments, the % outer surface coverage is from about 70% to about 80%. In embodiments, the % outer surface coverage is from about 70% to about 75%.

In embodiments, the % outer surface coverage is from about 75% to about 99%. In embodiments, the % outer surface coverage is from about 75% to about 95%. In embodiments, the % outer surface coverage is from about 75% to about 90%. In embodiments, the % outer surface coverage is from about 75% to about 85%. In embodiments, the % outer surface coverage is from about 75% to about 80%.

In embodiments, the % outer surface coverage is from about 80% to about 99%. In embodiments, the % outer surface coverage is from about 80% to about 95%. In embodiments, the % outer surface coverage is from about 80% to about 90%. In embodiments, the % outer surface coverage is from about 80% to about 85%.

In embodiments, the % outer surface coverage is from about 85% to about 99%. In embodiments, the % outer surface coverage is from about 85% to about 95%. In embodiments, the % outer surface coverage is from about 85% to about 90%.

In embodiments, the % outer surface coverage is from about 90% to about 99%.

In embodiments, the formulation is a colloidosome. In embodiments, the colloidosome is no more than 1000 nm in diameter. In embodiments, the colloidosome has a diameter of from about 50 nm to about 1000 nm. In embodiments, the colloidosome has a diameter of from about 60 nm to about 1000 nm. In embodiments, the colloidosome has a diameter of about 70 nm to about 1000 nm. In embodiments, the colloidosome has a diameter of about 80 nm to about 1000 nm. In embodiments, the colloidosome has a diameter of about 90 nm to about 1000 nm. In embodiments, the colloidosome has a diameter of about 100 nm to about 1000 nm.

In embodiments, the colloidosome has a diameter of from about 50 nm to about 900 nm. In embodiments, the colloidosome has a diameter of from about 60 nm to about 900 nm. In embodiments, the colloidosome has a diameter of about 70 nm to about 900 nm. In embodiments, the colloidosome has a diameter of about 80 nm to about 900 nm. In embodiments, the colloidosome has a diameter of about 90 nm to about 900 nm. In embodiments, the colloidosome has a diameter of about 100 nm to about 900 nm.

In embodiments, the colloidosome has a diameter of from about 50 nm to about 800 nm. In embodiments, the colloidosome has a diameter of from about 60 nm to about 800 nm. In embodiments, the colloidosome has a diameter of about 70 nm to about 800 nm. In embodiments, the colloidosome has a diameter of about 80 nm to about 800 nm. In embodiments, the colloidosome has a diameter of about 90 nm to about 800 nm. In embodiments, the colloidosome has a diameter of about 100 nm to about 800 nm.

In embodiments, the colloidosome has a diameter of from about 50 nm to about 700 nm. In embodiments, the colloidosome has a diameter of from about 60 nm to about 700 nm. In embodiments, the colloidosome has a diameter of about 70 nm to about 700 nm. In embodiments, the colloidosome has a diameter of about 80 nm to about 700 nm. In embodiments, the colloidosome has a diameter of about 90 nm to about 700 nm. In embodiments, the colloidosome has a diameter of about 100 nm to about 700 nm.

In embodiments, the colloidosome has a diameter of from about 50 nm to about 600 nm. In embodiments, the colloidosome has a diameter of from about 60 nm to about 600 nm. In embodiments, the colloidosome has a diameter of about 70 nm to about 600 nm. In embodiments, the colloidosome has a diameter of about 80 nm to about 600 nm. In embodiments, the colloidosome has a diameter of about 90 nm to about 600 nm. In embodiments, the colloidosome has a diameter of about 100 nm to about 600 nm.

In embodiments, the formulation is a colloidosome. In embodiments, the colloidosome is no more than 500 nm in diameter. In embodiments, the colloidosome has a diameter of from about 50 nm to about 500 nm. In embodiments, the colloidosome has a diameter of from about 60 nm to about 500 nm. In embodiments, the colloidosome has a diameter of about 70 nm to about 500 nm. In embodiments, the colloidosome has a diameter of about 80 nm to about 500 nm. In embodiments, the colloidosome has a diameter of about 90 nm to about 500 nm. In embodiments, the colloidosome has a diameter of about 100 nm to about 500 nm.

In embodiments, the colloidosome is no more than 400 nm in diameter. In embodiments, the colloidosome has a diameter of from about 50 nm to about 400 nm. In embodiments, the colloidosome has a diameter of from about 60 nm to about 400 nm. In embodiments, the colloidosome has a diameter of about 70 nm to about 400 nm. In embodiments, the colloidosome has a diameter of about 80 nm to about 400 nm. In embodiments, the colloidosome has a diameter of about 90 nm to about 400 nm. In embodiments, the colloidosome has a diameter of about 100 nm to about 400 nm.

In embodiments, the colloidosome is no more than 300 nm in diameter. In embodiments, the colloidosome has a diameter of from about 50 nm to about 300 nm. In embodiments, the colloidosome has a diameter of from about 60 nm to about 300 nm. In embodiments, the colloidosome has a diameter of about 70 nm to about 300 nm. In embodiments, the colloidosome has a diameter of about 80 nm to about 300 nm. In embodiments, the colloidosome has a diameter of about 90 nm to about 300 nm. In embodiments, the colloidosome has a diameter of about 100 nm to about 300 nm.

In embodiments, the colloidosome has a diameter of no more than 200 nm. In embodiments, the colloidosome has a diameter of from about 50 nm to about 200 nm. In embodiments, the colloidosome has a diameter of from about 60 nm to about 200 nm. In embodiments, the colloidosome has a diameter of about 70 nm to about 200 nm. In a preferred embodiment, the colloidosome has a diameter of about 80 nm to about 200 nm. In embodiments, the colloidosome has a diameter of about 90 nm to about 200 nm. In embodiments, the colloidosome has a diameter of about 100 nm to about 200 nm.

In embodiments, the colloidosome has a diameter of about 80 nm. In embodiments, the colloidosome has a diameter of about 90 nm. In embodiments, the colloidosome has a diameter of about 100 nm. In embodiments, the colloidosome has a diameter of about 110 nm. In embodiments, the colloidosome has a diameter of about 120 nm. In embodiments, the colloidosome has a diameter of about 130 nm. In embodiments, the colloidosome has a diameter of about 140 nm. In embodiments, the colloidosome has a diameter of about 150 nm.

In embodiments, the % outer surface coverage of the microgel by the nanofibrils

$$\left(\frac{C}{{C}_{s a t}}\right)$$

is calculated by the following equation:

$$\frac{3 c}{{c}_{s a t}} = - l n \left(\frac{{ζ}_{c} - {ζ}_{s a t}}{{ζ}_{o} - {ζ}_{s a t}}\right)$$

wherein: ζₛₐₜ is the ζ-potential when the microgels are saturated with biopolymeric nanofibrils; ζ₀ is he ζ-potential of the proteinaceous or non-proteinaceous microgel in absence of the biopolymeric nanofibrils; and ζ_{c} is the ζ-potential of the formulation (i.e., the colloidosome) at biopolymeric nanofibril concentration c. cₛₐₜ is the minimum amount of the biopolymeric nanofibrils required to completely cover the surface of the proteinaceous or non-proteinaceous microgel [Anges Teo, Sung Je Lee, Kelvin K. T. Goh, Food Structure, 2017, 14, 60-67; Anwesha Sarkar, Kelvin K.T. Goh, Harjinder Singh, Food Hydrocolloids, 2009, 23, 1270-1278; S. Pallandre, E. A. Decker and D. J. McClements, Journal of Food Science, 2007, 72, E518-E524; Demet Guzey and David Julian McClements, J. Agric. Food Chem., 2007, 55, 475-485].

The weight ratio of one or more biopolymeric nanofibrils to proteinaceous or non-proteinaceous microgel may be from about 0.01:1 to about 10:1. In embodiments, the weight ratio is from about 0.01:1 to about 5:1. In embodiments, the weight ratio is from about 0.01:1 to about 4:1. In embodiments, the weight ratio is from about 0.01:1 to about 3:1. In embodiments, the weight ratio is from about 0.01:1 to about 2:1. In embodiments, the weight ratio is from about 0.01:1 to about 1.5:1. In embodiments, the weight ratio is from about 0.01:1 to about 1:1.

The weight ratio of one or more biopolymeric nanofibrils to proteinaceous or non-proteinaceous microgel may be from about 0.1:1 to about 10:1. In embodiments, the weight ratio is from about 0.1:1 to about 5:1. In embodiments, the weight ratio is from about 0.1:1 to about 4:1. In embodiments, the weight ratio is from about 0.1:1 to about 3:1. In embodiments, the weight ratio is from about 0.1:1 to about 2:1. In embodiments, the weight ratio is from about 0.1:1 to about 1.5:1. In embodiments, the weight ratio is from about 0.1:1 to about 1:1.

In embodiments, the weight ratio of one or more biopolymeric nanofibrils to proteinaceous or non-proteinaceous microgel is from about 0.2:1 to about 5:1. In embodiments, the weight ratio is from about 0.3:1 to about 5:1. In embodiments, the weight ratio is from about 0.4:1 to about 5:1. In embodiments, the weight ratio is from about 0.5:1 to about 5:1. In embodiments, the weight ratio is from about 0.6:1 to about 5:1. In embodiments, the weight ratio is from about 0.7:1 to about 5:1. In embodiments, the weight ratio is from about 0.8:1 to about 5:1. In embodiments, the weight ratio is from about 0.9:1 to about 5:1. In embodiments, the weight ratio is from about 1:1 to about 5:1. In embodiments, the weight ratio is from about 1.5:1 to about 5:1. In embodiments, the weight ratio is from about 2:1 to about 5:1. In embodiments, the weight ratio is from about 3:1 to about 5:1. In embodiments, the weight ratio is from about 4:1 to about 5:1.

In embodiments, the weight ratio of one or more biopolymeric nanofibrils to proteinaceous or non-proteinaceous microgel is from about 0.2:1 to about 3:1. In embodiments, the weight ratio of one or more biopolymeric nanofibrils to proteinaceous or non-proteinaceous microgel is from about 0.2:1 to about 2:1. In embodiments, the weight ratio is from about 0.3:1 to about 2:1. In embodiments, the weight ratio is from about 0.4:1 to about 2:1. In embodiments, the weight ratio is from about 0.5:1 to about 2:1. In embodiments, the weight ratio is from about 0.6:1 to about 2:1.

In embodiments, the weight ratio of one or more biopolymeric nanofibrils to proteinaceous or non-proteinaceous microgel is from about 0.6:1 to about 1.5:1. In embodiments, the weight ratio is from about 0.6:1 to about 1:1.

In a preferred embodiment, the weight ratio is from about 0.2:1 to about 3:1.

In a preferred embodiment, the weight ratio is from about 0.6:1 to about 2:1.

In an embodiment, the formulation further comprises a pharmaceutically acceptable excipient. In an embodiment, the pharmaceutically acceptable excipient comprises a buffered solution having a pH of from about 3.0 to about 7.0. In an embodiment, the pharmaceutically acceptable excipient comprises a buffered solution having a pH of from about 3.0 to about 4.0. In an embodiment, the pharmaceutically acceptable excipient comprises a buffered solution having a pH of about 7.0.

In a preferred embodiment, the microgel is lactoferrin and the one or more biopolymeric nanofibrils are κ-carrageenan nanofibrils. In a preferred embodiment, the microgel is lactoferrin and the one or more biopolymeric nanofibrils are made by addition of agar. In a preferred embodiment, the microgel is potato protein and the one or more biopolymeric nanofibrils are κ-carrageenan nanofibrils. In a preferred embodiment, the microgel is potato protein and the one or more biopolymeric nanofibrils are made by addition of xanthan gum.

### Method for Preparing a Formulation

In embodiments, the buffer solution of step (a) may be selected from the group consisting of: 4-(2-hydroxyethyl)-1-piperazineethanesulfonic acid (HEPES), phosphate buffer, 2-(N-Morpholino)ethanesulfonic acid hydrate, 4-Morpholineethanesulfonic acid (MES hydrate), 2,2-Bis(hydroxymethyl)-2,2′,2″-nitrilotriethanol (Bis-Tris), citric acid monohydrate and trisodium citrate dihydrate.

In embodiments, the buffer solution of step (a) may be selected from the group consisting of: 4-(2-hydroxyethyl)-1-piperazineethanesulfonic acid (HEPES), phosphate buffer, 2-(N-Morpholino)ethanesulfonic acid hydrate, 4-Morpholineethanesulfonic acid (MES hydrate), 2,2-Bis(hydroxymethyl)-2,2′,2″-nitrilotriethanol (Bis-Tris) and citric acid monohydrate.

In embodiments, the buffer solution of step (a) has a concentration of from about 1 to 50 mM. In an embodiment, the buffer solution of step (a) has a concentration of about 20 mM. In a preferred embodiment, the buffer solution of step (a) has a concentration of about 10 mM.

In embodiments, the buffer solution of step (a) has a pH of from about 3.0 to about 7.0. In an embodiment, the buffer solution of step (a) has a pH of from about 3.0 to about 4.0. In a preferred embodiment, the buffer solution of step (a) has a pH of about 7.0. In a preferred embodiment, the buffer solution of step (a) has a pH of about 3.0.

In embodiments, the proteinaceous or non-proteinaceous microgel is selected from the group consisting of: lactoferrin, lysozyme, gelatin, milk protein, bovine serum albumin, whey protein, casein, caseinate, egg protein, albumin, gluten, gelatin Type B, pea protein, rice protein, legumin, corn protein, peanut protein, chitosan, chitin and potato protein.

In embodiments, the proteinaceous or non-proteinaceous microgel is selected from the group consisting of: lactoferrin, lysozyme, gelatin, milk protein, bovine serum albumin, whey protein, caseinate, egg protein, albumin, gluten, gelatin Type B, pea protein, rice protein, legumin, corn protein, peanut protein, chitosan and chitin.

In embodiments, the material is a proteinaceous microgel. This embodiment is the preferred embodiment of the present invention. In embodiments, the proteinaceous microgel is selected from the group consisting of: lactoferrin, lysozyme, gelatin, milk protein, bovine serum albumin, whey protein, casein, caseinate, egg protein, albumin, gluten, gelatin Type B, pea protein, rice protein, legumin, corn protein, peanut protein and potato protein. In embodiments, the proteinaceous microgel is selected from the group consisting of: lactoferrin, lysozyme, gelatin, milk protein, bovine serum albumin, whey protein, caseinate, egg protein, albumin, gluten, gelatin Type B, pea protein, rice protein, legumin, corn protein, peanut protein. In a preferred embodiment, the microgel is lactoferrin. In a preferred embodiment, the microgel is potato protein.

In embodiments, the microgel is a non-proteinaceous microgel. In embodiments, the non-proteinaceous microgel is selected from the group consisting of: chitosan and chitin.

In embodiments, the resulting solution of step (a) comprises the proteinaceous or non-proteinaceous material in an amount of at least about 4 wt%. In embodiments, the resulting solution of step (a) comprises the proteinaceous or non-proteinaceous material in an amount of at least about 6 wt%. In embodiments, the resulting solution of step (a) comprises the proteinaceous or non-proteinaceous material in an amount of at least about 8 wt%. In embodiments, the resulting solution of step (a) comprises the proteinaceous or non-proteinaceous material in an amount of no more than about 20 wt%.

In a preferred embodiment, the resulting solution of step (a) comprises the proteinaceous or non-proteinaceous material in an amount of about 12 wt%. In a preferred embodiment, the resulting solution of step (a) comprises the proteinaceous or non-proteinaceous material in an amount of about 9 wt%. In a preferred embodiment, the resulting solution of step (a) comprises the proteinaceous or non-proteinaceous material in an amount of about 6 wt%.

In embodiments, dissolving the proteinaceous or non-proteinaceous material in the buffer solution in step (a) comprises stirring the mixture until complete solubilisation occurs. In embodiments, dissolving the proteinaceous or non-proteinaceous material in the buffer solution in step (a) involves stirring the mixture for at least about 5 minutes, for at least about 20 minutes, for at least about 30 minutes, for at least about 40 minutes, for at least about 50 minutes, for at least about 1 hour, for at least about 1.5 hours, for at least about 2 hours, or for at least about 2.5 hours.

In a preferred embodiment, dissolving the proteinaceous or non-proteinaceous material in the buffer solution in step (a) involves stirring the mixture for about 2 hours.

In embodiments, heating the resulting solution in step (a) is performed for at least about 10 minutes, for at least about 20 minutes or for at least about 30 minutes.

In a preferred embodiment, heating the resulting solution in step (a) is performed for about 30 minutes.

In embodiments, heating the resulting solution in step (a) is performed at a temperature of at least about 65° C. (e.g., at least about 65° C., at least about 70° C., at least about 75° C., or at least about 80° C.). In embodiments, heating the resulting solution in step (a) is performed at a temperature of at least about 70° C. (e.g., at least about 75° C., at least about 80° C., at least about 85° C., or at least about 90° C.). In embodiments, heating the resulting solution in step (a) is performed at a temperature of at least about 65° C. and no more than about 150° C. (e.g., at least about 65° C. and no more than about 140° C., at least about 70° C. and no more than about 130° C. or at least about 80° C. and no more than about 110° C. In embodiments, heating the resulting solution in step (a) is performed at a temperature of at least about 70° C. and no more than about 150° C. (e.g., at least about 70° C. and no more than about 140° C., at least about 80° C. and no more than about 130° C. or at least about 90° C. and no more than about 110° C.

In a preferred embodiment, heating the resulting solution is performed at about 90° C. In a preferred embodiment, heating the resulting solution is performed at about 65° C.

In embodiments, the weight ratio of heat-set gel to buffer solution in step (b) is about 3:1 w/w.

In embodiments, the step of homogenising to form the proteinaceous or non-proteinaceous microgel in step (b) is performed at a pressure of at least 300 bar.

In embodiments, step (b) further comprises the step of blending the mixture of heat-set gel and buffer solution to form macrogel particles before homogenising to form the proteinaceous or non-proteinaceous microgel.

In embodiments, step (b) further comprises the step of degassing the mixture of heat-set gel and buffer solution before homogenising to form the proteinaceous or non-proteinaceous microgel. In such embodiments, the mixture is degassed for at least about 3 minutes.

In embodiments, the solution of one or more biopolymeric nanofibrils of step (c) comprises at least about 0.05 wt% of the one or more biopolymeric nanofibrils. In embodiments, the solution of one or more biopolymeric nanofibrils of step (c) comprises no more than about 5 wt% of the one or more biopolymeric nanofibrils. In embodiments, the solution of one or more biopolymeric nanofibrils of step (c) comprises from about 0.05 wt% to about 3 wt % of the one or more biopolymeric nanofibrils.

In a preferred embodiment, the solution of one or more biopolymeric nanofibrils of step (c) comprises about 1.5 wt% of the one or more biopolymeric nanofibrils.

In embodiments, the buffer solution of step (c) may be selected from the group consisting of: 4-(2-hydroxyethyl)-1-piperazineethanesulfonic acid (HEPES), phosphate buffer, 2-(N-Morpholino)ethanesulfonic acid hydrate, 4-Morpholineethanesulfonic acid (MES hydrate), 2,2-Bis(hydroxymethyl)-2,2′,2″-nitrilotriethanol (Bis-Tris), citric acid monohydrate and trisodium citrate dihydrate.

In embodiments, the solution of one or more biopolymeric nanofibrils of step (c) comprises a buffer solution. In such embodiments, the buffer solution may be selected from the group consisting of: 4-(2-hydroxyethyl)-1-piperazineethanesulfonic acid (HEPES), phosphate buffer, 2-(N-Morpholino)ethanesulfonic acid hydrate, 4-Morpholineethanesulfonic acid (MES hydrate), 2,2-Bis(hydroxymethyl)-2,2′,2″-nitrilotriethanol (Bis-Tris) and citric acid monohydrate.

In such embodiments, the buffer solution of step (c) has a concentration of from about 1 mM to 50 mM. In an embodiment, the buffer solution of step (c) has a concentration of about 20 mM. In a preferred embodiment, the buffer solution of step (c) has a concentration of about 10 mM.

In embodiments, the weight ratio of the proteinaceous or non-proteinaceous microgel to one or more biopolymeric nanofibrils used in step (c) is selected in accordance with paragraphs [0086] to [0092] the first and second aspects of the invention.

In embodiments, the solution of one or more biopolymeric nanofibrils of step (c) is formed by (i) heating a mixture of one or more biopolymeric materials and buffer solution while shearing the mixture to form the one or more biopolymeric nanofibrils, and (ii) cooling the resulting aqueous dispersion comprising the nanofibrils. In such embodiments, heating the mixture in step (i) may be performed at a temperature of at least about 50° C. (e.g., at least about 60° C., at least about 70° C., at least about 80° C., at least about 90° C.). In such embodiments, cooling the resulting aqueous dispersion comprising the one or more biopolymeric nanofibrils in step (ii) may be performed at around 37° C.

### Uses

In an aspect of the invention, there is provided a use of the formulation of the invention as a lubricant food additive, i.e. for fat replacement purposes. In embodiments, the use includes applying a formulation of the invention to food in a concentration of from about 5 to about 90%.

In embodiments, the use involves the addition of the lubricant food additive to a beverage or solid food selected from the group consisting of: chewing gum, candy, chocolate and frozen food products.

### EXAMPLES

### Materials and Methods

Lactoferrin was purchased from Ingredia, France; κ-carrageenan was purchased from Sigma-Aldrich, UK; agar was purchased from Scientific Laboratory Supplies, UK; potato protein was purchased from Sosa Ingredients, Spain; xanthan gum was purchased from Sigma-Aldrich, UK. Biopolymers, lactoferrin, potato protein isolate, κ-carrageenan, agar and xanthan gum were made in pH 7.0 buffer consisting of 10 mM 4-(2-hydroxyethyl)-1-piperazineethanesulfonic acid (HEPES), the pH was adjusted to salivary pH (pH 7.0) by adding NaOH, or in pH 3.0 buffer consisting of 10 mM citric acid monohydrate and 10 mM trisodium citrate dihydrate mixed in adequate proportions so as to reach an acidic pH (pH 3.0). Milli-Q water purified by treatment with a Milli-Q apparatus (Millipore Corp., Bedford, MA, USA) was used to prepare the buffer.

### Characterisation

### ζ-Potential Measurement

The ζ-potential of samples at pH 7.0 or pH 3.0 were measured by Zetasizer (Nano ZS series, Malvern Instruments Ltd., UK). The samples were added into folded electrophoretic cells (DTS1070, Malvern Instruments Ltd., UK) at 25° C. and diluted 100 times before the measurement.

### Transmission Electron Microscopy

Transmission electron microscopy of samples was performed using a transmission electron microscope (Tecnai G2 Spirit-T12, ThermoFisher, USA). Voltage of the electron gun was fixed at 120 kV and images were captured using a Gantan CCD camera. In order to increase the electron contrast, the samples were negatively stained. For this purpose, 5.0 µL of the samples were deposited on a carbon coated TEM grid. Before depositing the samples, the grid was electrostatically cleaned using a Pelco easyGlow discharge cleaning system (Ted Pella, Inc., USA). After deposition, the samples were left to rest for 60 s and excess of the sample at the edge of the grid was removed using a filter paper. Samples were stained by adding 5.0 µL of 1.0% uranyl acetate for 10 s and the excess of uranyl acetate was removed. The treatment with uranyl acetate was repeated twice and the samples were then air-dried before imaging.

### Rheology

A modular compact controlled-stress rheometer (MCR-302, Anton Paar, Austria) was used to measure the apparent viscosity of all samples, equipped with a cone-plate geometry (CP50-1, diameter 50 mm, angle 1°). The gap size corresponding to this geometry was 0.208 mm. Viscosity measurements were performed in a range of shear rates from 0.1 s⁻¹ to 100 s⁻¹ at a fixed temperature of 37° C. The data points were set to be 6 points/decade, and the duration was set by the device to ensure reaching steady state for each point. In addition, the rheology of real human saliva (MEEC 16-046, ethics approved by Faculty Ethics Committee, University of Leeds) and honey was used as controls.

### Tribology

A Mini Traction Machine (MTM2, PCS Instruments, UK) was used to measure the lubrication properties of all samples, with hydrophobic polydimethylsiloxane (PDMS) ball (Ø 19 mm)-on-disk (Ø 46 mm) configuration mimicking the hydrophobic tongue-palate of dry mouth. The surface roughness Rₐ of PDMS (Sylgard 184, Dow Corning, USA) was 50 nm. The temperature was set at 37° C. and the load was fixed at 2.0 N for all experiments. In addition, the tribology of real human saliva (MEEC 16-046, ethics approved by Faculty Ethics Committee, University of Leeds) was used as controls.

With this invention, the inventors demonstrate formulations comprising proteinaceous or non-proteinaceous microgels partially coated with polysaccharide-based nanofibrils. These formulations achieve better lubrication performance than commercial lubricants and human saliva, and provide lowering of friction coefficients without the need of high viscosity.

### Example 1: Manufacture of Lactoferrin Microgels Coated by Κ-Carrageenan Nanofibrils

### KCnF/LFM - 140 Nm

κ-carrageenan nanofibrils (KCnF) were prepared by dissolving κ-carrageenan powder in 10 mM HEPES buffer (mentioned above) by heating at 95° C. while being sheared for 40 minutes under constant stirring for a complete solubilisation and formation of nanofibrils. This aqueous dispersion containing KCnF was then cooled to around 37° C.

Lactoferrin solution (12 wt%) was prepared by adding lactoferrin powder in 10 mM HEPES buffer at pH 7.0 and stirring for 2 hours to ensure complete solubilisation. The solution was heated at 90° C. for 30 minutes to form heat-set gel, which was mixed with 10 mM HEPES buffer (3:1 w/w) at pH 7.0 and broken into macrogel particles using a hand blender (HB724, Kenwood, UK) for 5 minutes. Then the resulting lactoferrin macrogel particles + buffer mixture was transferred to a conditioning mixer (ARE-250, THINKY Corporation, Japan) for degassing for 3 minutes. The degassed macrogel particle + buffer mixture was then homogenized by passing twice through Leeds Jet Homogenizer operating at a pressure of 300 ± 20 bars to form lactoferrin microgel (LFM) particles.

The formulation was prepared by adding LFM to KCnF under gentle stirring at different weight ratios ranging from 0.01:1 to 3:1 w/w (KCnF/LFM).

The different weight ratios are illustrated in the following Table:

| KCnF in formulation (wt%) | LFM in formulation (wt%) | Ratio of KCnF/LFM (wt/wt) |
|---|---|---|
| 0.02 | 2.00 | 0.01 |
| 0.07 | 2.00 | 0.03 |
| 0.14 | 2.00 | 0.07 |
| 0.40 | 2.00 | 0.20 |
| 0.80 | 2.00 | 0.40 |
| 1.16 | 2.00 | 0.60 |
| 1.16 | 1.16 | 1.00 |
| 1.16 | 0.58 | 2.00 |
| 1.16 | 0.39 | 3.00 |

### Example 2: Manufacture of Lactoferrin Microgels Coated by Κ-Carrageenan Nanofibrils

### KCnF/LFM - 90 Nm

κ-carrageenan nanofibrils (KCnF) were prepared by dissolving κ-carrageenan powder in 10 mM HEPES buffer (mentioned above) at pH 7.0 by heating at 95° C. while being sheared for 40 minutes under constant stirring for a complete solubilisation and formation of nanofibrils. This aqueous dispersion containing KCnF was then cooled to around 37° C.

Lactoferrin solution (9 wt%) was prepared by adding lactoferrin powder in 10 mM HEPES buffer at pH 7.0 and stirring for 2 hours to ensure complete solubilisation. Then the solution was heated at 90° C. for 30 minutes to form lactoferrin microgel (LFM) particles.

The formulation was prepared by adding LFM to KCnF under gentle stirring at different weight ratios ranging from 0.01:1 to 2:1 w/w (KCnF/LFM). The different weight ratios are illustrated in the following Table:

| KCnF in formulation (wt%) | LFM in formulation (wt%) | Ratio of KCnF/LFM (wt/wt) |
|---|---|---|
| 0.02 | 2.00 | 0.01 |
| 1.16 | 2.00 | 0.60 |
| 1.16 | 1.16 | 1.00 |
| 1.16 | 0.58 | 2.00 |

### Example 3: Manufacture of Lactoferrin Microgels Coated by Agar Nanofibrils

### AnF/LFM - 90 Nm

Agar nanofibrils (AnF) were prepared by dissolving agar powder in 10 mM HEPES buffer (mentioned above) at pH 7.0 by heating at 95° C. while being sheared for 40 minutes under constant stirring for a complete solubilisation and formation of nanofibrils. This aqueous dispersion containing AnF was then cooled to around 37° C.

Lactoferrin solution (9 wt%) was prepared by adding lactoferrin powder in 10 mM HEPES buffer at pH 7.0 and stirring for 2 hours to ensure complete solubilisation. Then the solution was heated at 90° C. for 30 minutes to form lactoferrin microgel (LFM) particles.

The formulation was prepared by adding LFM to AnF under gentle stirring at different weight ratios ranging from 0.01:1 to 1:1 w/w (AnF/LFM). The different weight ratios are illustrated in the following Table:

| AnF in formulation (wt%) | LFM in formulation (wt%) | Ratio of AnF/LFM (wt/wt) |
|---|---|---|
| 0.02 | 2.00 | 0.01 |
| 1.16 | 1.16 | 1.00 |

### Example 4: Manufacture of Potato Protein Microgels Coated by Κ-Carrageenan Nanofibrils

### KCnF/PoPM - 100 Nm

κ-carrageenan nanofibrils (KCnF) were prepared by dissolving κ-carrageenan powder in 10 mM citrate buffer (mentioned above) at pH 3.0 by heating at 95° C. while being sheared for 40 minutes under constant stirring for a complete solubilisation and formation of nanofibrils. This aqueous dispersion containing KCnF was then cooled to around 37° C.

Potato protein isolate solution (6 wt%) was prepared by adding potato protein isolate powder in 10 mM citrate buffer at pH 3.0 and stirring for 2 hours to ensure complete solubilisation. Then the pH of the solution was adjusted to 3.0 by adding HCl and finally the solution was heated at 65° C. for 30 minutes to form potato protein microgel (PoPM) particles.

The formulation was prepared by adding PoPM to KCnF under gentle stirring at different weight ratios ranging from 0.01:1 to 2:1 w/w (KCnF/PoPM). The different weight ratios are illustrated in the following Table:

| KCnF in formulation (wt%) | PoPM in formulation (wt%) | Ratio of KCnF/PoPM (wt/wt) |
|---|---|---|
| 0.02 | 2.00 | 0.01 |
| 1.00 | 2.00 | 0.50 |
| 1.16 | 0.58 | 2.00 |

### Example 5: Manufacture of Potato Protein Microgels Coated by Xanthan Gum Nanofibrils

### XGnF/PoPM - 100 Nm

Xanthan gum nanofibrils (XGnF) were prepared by dissolving xanthan gum powder in 10 mM citrate buffer at pH 3.0 (mentioned above) at room temperature while being sheared for 24 hours under constant stirring for a complete solubilisation, hydration and formation of nanofibrils.

Potato protein isolate solution (6 wt%) was prepared by adding potato protein isolate powder in 10 mM citrate buffer at pH 3.0 and stirring for 2 hours to ensure complete solubilisation. Then the pH of the solution was adjusted to 3.0 by adding HCl and finally the solution was heated at 65° C. for 30 minutes to form potato protein microgel (PoPM) particles.

The formulation was prepared by adding PoPM to XGnF under gentle stirring at different weight ratios ranging from 0.01:1 to 2:1 w/w (KCnF/PoPM). The different weight ratios are illustrated in the following Table:

| XGnF in formulation (wt%) | PoPM in formulation (wt%) | Ratio of XGnF/PoPM (wt/wt) |
|---|---|---|
| 0.02 | 2.00 | 0.01 |
| 1.00 | 2.00 | 0.50 |
| 1.16 | 0.58 | 2.00 |

### Example 6: Analysis of Lactoferrin Microgels Coated by Κ-Carrageenan Nanofibrils Manufactured in Example 1

A ratio of KCnF : LFM of 0.6 : 1 was selected for the lactoferrin microgels coated by κ-carrageenan nanofibrils used in the following analysis.

The transmission electron micrographs in FIG. 1a show LFM particles as circular dark areas with diameters of less than 300 nm. In FIG. 1b, KCnF show an average diameter and length of 10-20 nm and 100-300 nm, respectively. FIGS. 1c and 1d show LFM particles covered by KCnF. KCnF are also seen in the continuous phase connecting different colloidosome subunits.

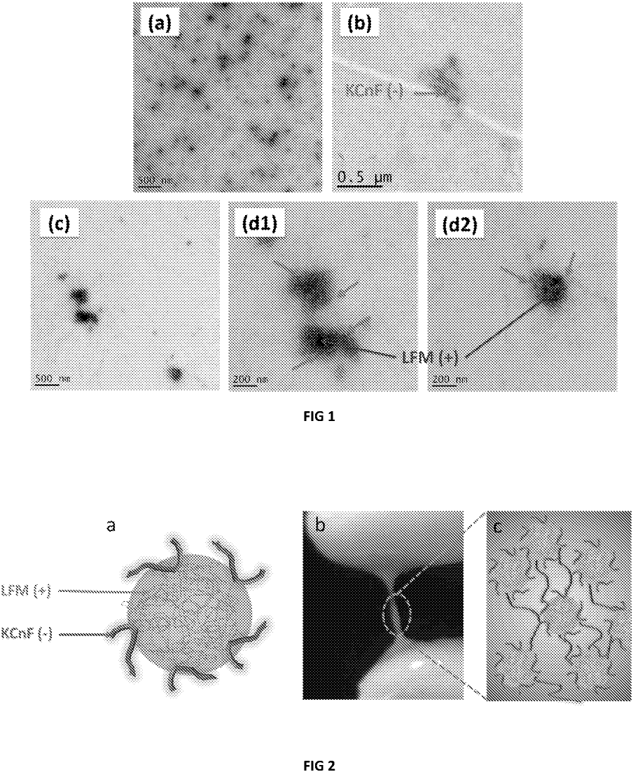

FIG. 2a shows the schematic representation of the colloidosome composed by LFM particles that are coated by KCnF. Under uniaxial tensile deformation, the colloidosome forms a macroscopic filament spanning the surfaces applying the deformation (FIGS. 2b and 2c). This kind of structure under tensile testing is commonly shown by polymer melts and solutions, and is an important feature of human saliva.

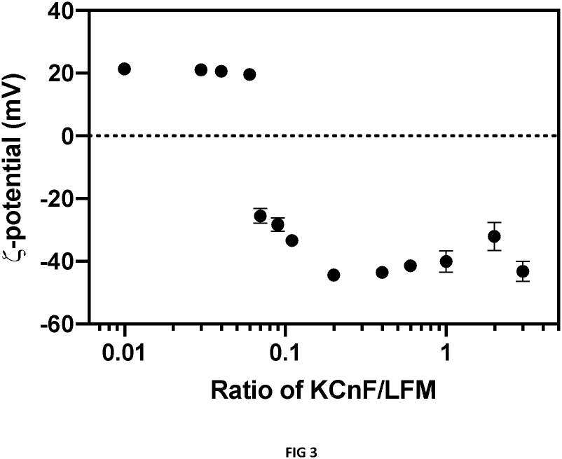

The ζ-potential decreases upon increasing the concentration of KCnF (negatively charged) relatively to the concentration of LFM (positively charged) (FIG. 3). Raising the ratio, i.e., increasing the concentration of KCnF, allows the transition of the ζ-potential from positive to negative. In other words, upon increasing KCnF/LFM ratio, LFM particles become gradually negatively charged due to the gradual coverage by KCnF as shown in the table below:

ζ-potential of LFM, KCnF and colloidosomes

| | LFM | KCnF | KCnF/LFM (0.6:1 w/w) |
|---|---|---|---|
| ζ-potential (mV) | +22.0 | -46.3 | -41.5 |

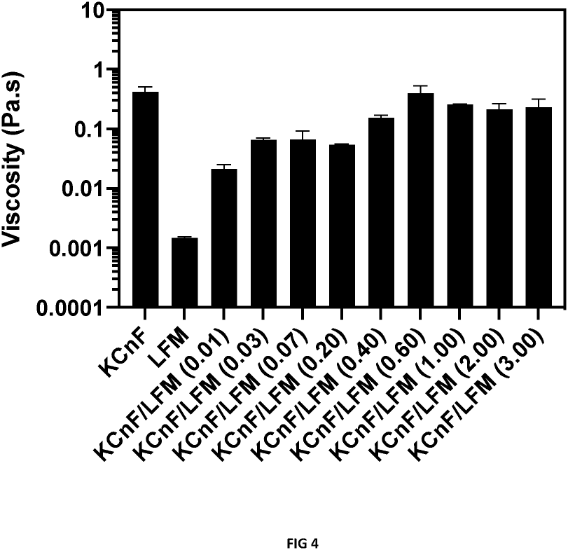

To evaluate the fluidity of the colloidosomes under relevant oral conditions, the apparent viscosity of our samples at various ratios at an orally relevant shear rate was compared (FIG. 4). For comparative purposes, the fluidity of two of our samples was compared with real human saliva, honey and various commercially available formulations at an orally relevant shear rate (FIG. 5). In comparison to high viscosity fluid food products, such as honey and certain commercial formulations, the viscosity of the new colloidosome was one order of magnitude lower, indicating the good fluidity of the particle mixture.

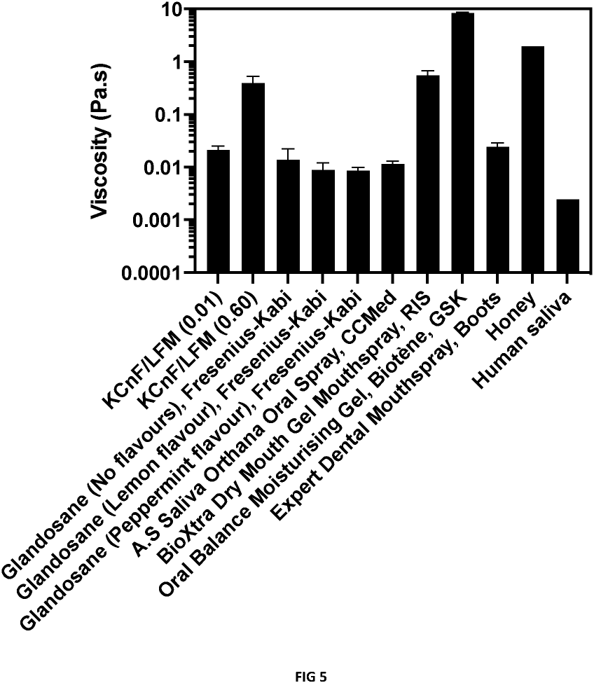

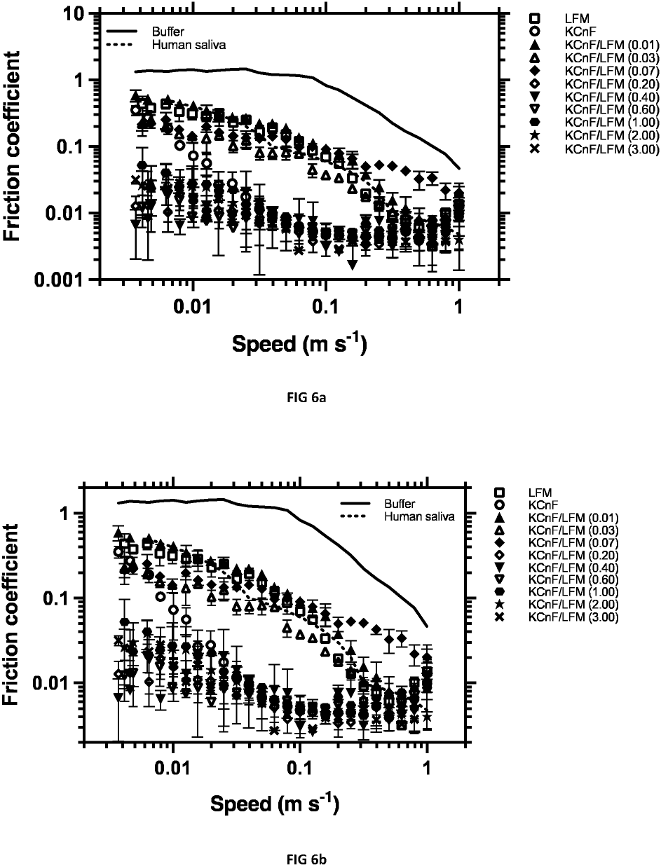

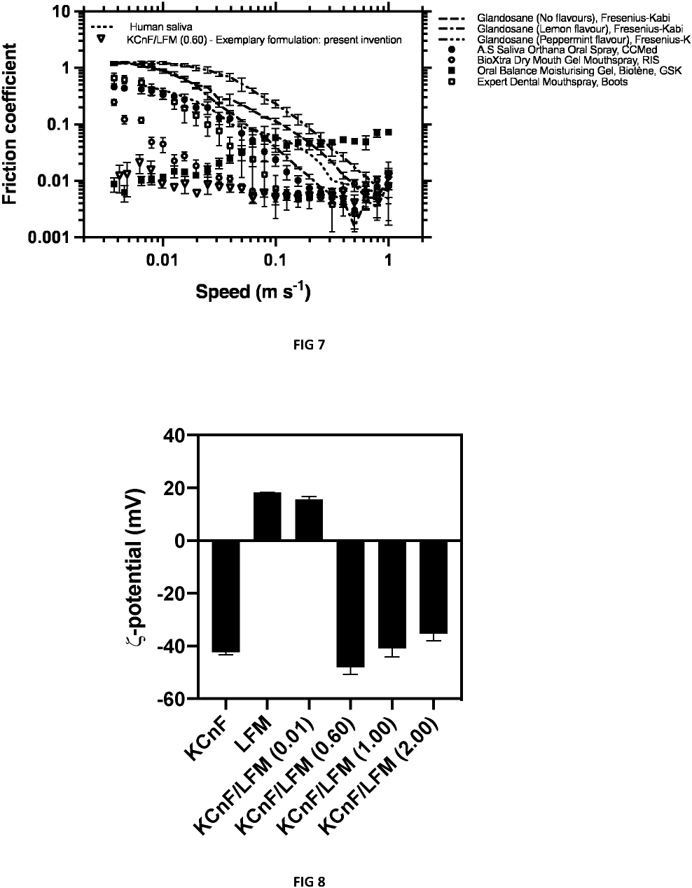

FIGS. 6 shows the lubrication performance of the different colloidosomes under orally relevant conditions, represented by the friction coefficient as a function of speed. For comparative purposes, the lubrication performance of two of our samples was compared with real human saliva, buffer and various commercially available formulations under orally relevant conditions (FIG. 7).

In comparison to buffer, both LFM and KCnF decrease the friction coefficient at orally relevant speeds ranging from 0.004 to 0.1 m/s by at least two folds (FIGS. 6). However, in comparison to real human saliva, friction coefficients obtained for both are twice as high in the boundary regime. A good salivary substitute is expected to surpass the tribological performance of real human saliva in both the boundary and fluid film regimes. Therefore, the reduction obtained by the components separately is not enough.

Additionally, the formulations of the invention demonstrate improved friction coefficients across all speeds tested unlike the commercially available lubricants (FIG. 7). At lower speeds, Biotène® Oral Balance Moisturising Gel has comparable friction coefficients to that exhibited by the present invention, but at higher speeds, friction coefficients are far worse than real human saliva. At higher speeds, BioXtra Dry Mouth Gel Mouthspray, Boots Expert Dental Mouthspray, A.S Saliva Orthana Oral Spray and some of Glandosane sprays have comparable friction coefficients to that exhibited by the present invention, but at lower speeds, friction coefficients are far worse.

### Summary:

The formulations of the present invention, i.e., KCnF-coated LFM, is capable to provide a reduction in friction coefficients in comparison to real human saliva, throughout the entire orally relevant speeds. However, on decreasing KCnF:LFM ratio, friction coefficients increase back higher than real human saliva, which is in agreement with the ζ-potential measurements (FIG. 3).

### Example 7: Analysis of Lactoferrin Microgels Coated by Κ-Carrageenan Nanofibrils Manufactured in Example 2

The ζ-potential decreases upon increasing the concentration of KCnF (negatively charged) relatively to the concentration of LFM (positively charged) (FIG. 8). Raising the ratio, i.e., increasing the concentration of KCnF, allows the transition of the ζ-potential from positive to negative. In other words, upon increasing KCnF/LFM ratio, LFM particles become gradually negatively charged due to the gradual coverage by KCnF.

To evaluate the fluidity of the colloidosomes under relevant oral conditions, the apparent viscosity of our samples at various ratios at an orally relevant shear rate was compared (FIG. 9).

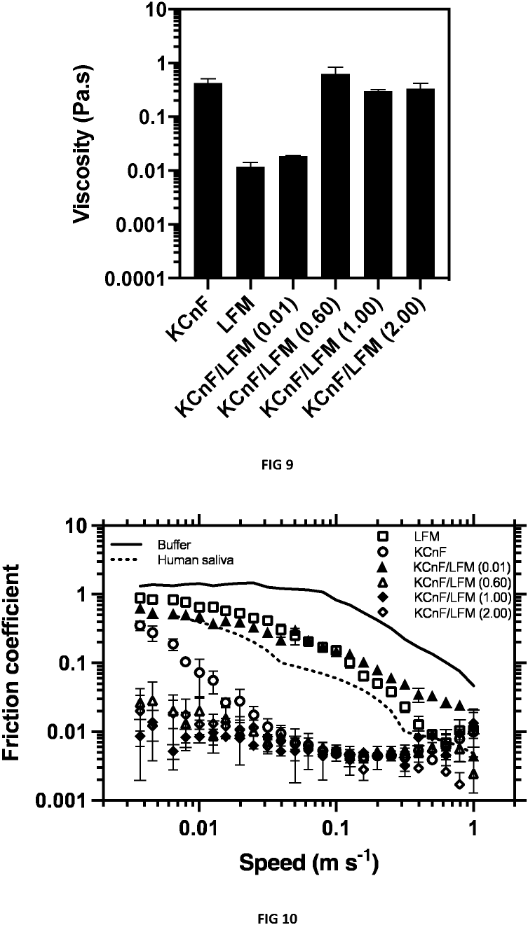

FIG. 10 shows the lubrication performance of the different colloidosomes under orally relevant conditions, represented by the friction coefficient as a function of speeds. The lubrication properties of buffer and real human saliva are also shown for comparison purposes.

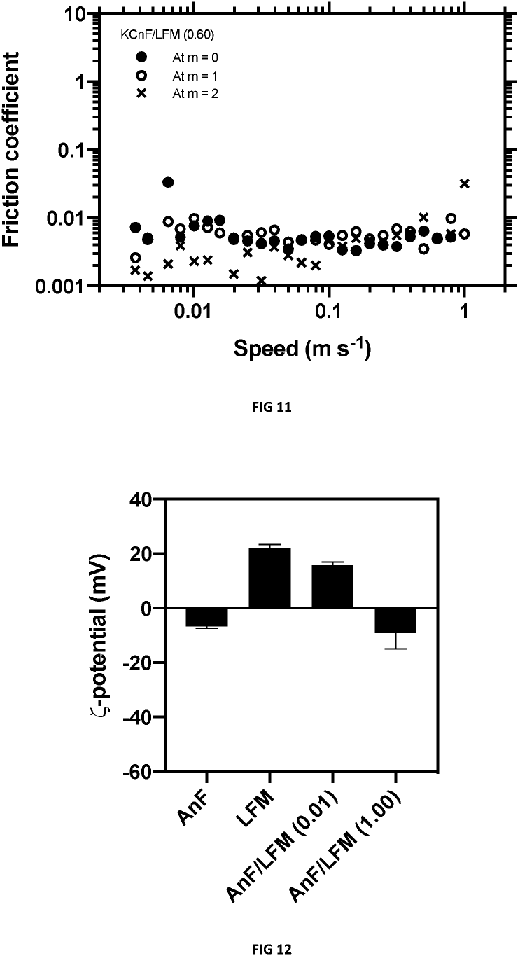

FIG. 11 shows the lubrication performance of the different colloidosomes under orally relevant conditions, represented by the friction coefficient as a function of speeds, at 0 month, 1 month and 2 months storage. This demonstrates that the lubricants are stable.

### Example 8: Analysis of Lactoferrin Microgels Coated by Agar Nanofibrils Manufactured in Example 3

The ζ-potential decreases upon increasing the concentration of AnF (negatively charged) relatively to the concentration of LFM (positively charged) (FIG. 12). Raising the ratio, i.e., increasing the concentration of AnF, allows the transition of the ζ-potential from positive to negative. In other words, upon increasing AnF/LFM ratio, LFM particles become gradually negatively charged due to the gradual coverage by AnF.

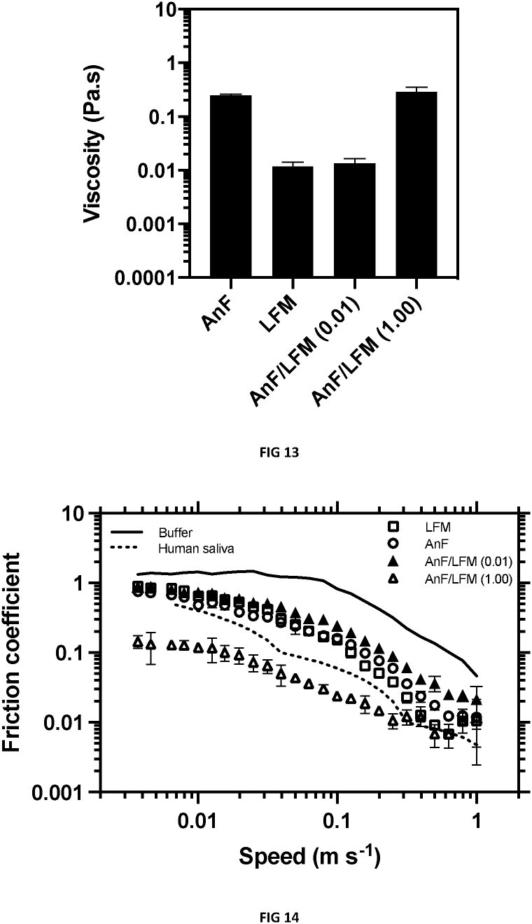

To evaluate the fluidity of the colloidosomes under relevant oral conditions, the apparent viscosity of our samples at various ratios at an orally relevant shear rate was compared (FIG. 13).

FIG. 14 shows the lubrication performance of the different colloidosomes under orally relevant conditions, represented by the friction coefficient as a function of speeds. The lubrication properties of buffer and real human saliva are also shown for comparison purposes.

### Example 9: Analysis of Potato Protein Microgels Coated by _{K-}Carrageenan nanofibrils Manufactured in Example 4

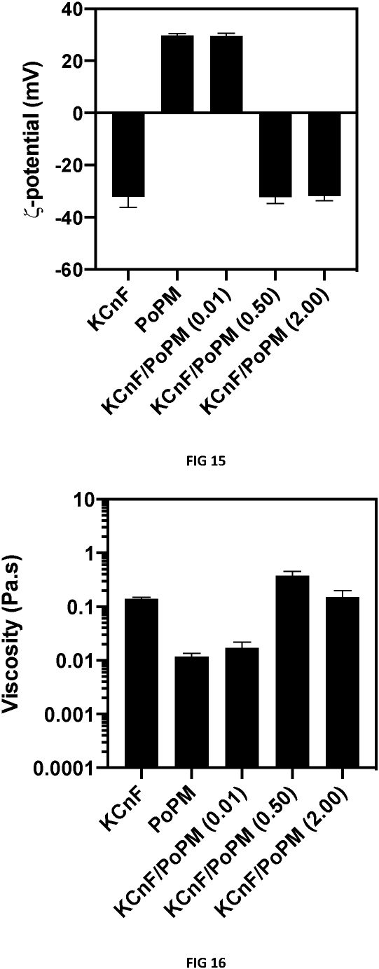

The ζ-potential decreases upon increasing the concentration of KCnF (negatively charged) relatively to the concentration of PoPM (positively charged) (FIG. 15). Raising the ratio, i.e., increasing the concentration of KCnF, allows the transition of the ζ-potential from positive to negative. In other words, upon increasing KCnF/PoPM ratio, PoPM particles become gradually negatively charged due to the gradual coverage by KCnF.

To evaluate the fluidity of the colloidosomes under relevant oral conditions, the apparent viscosity of our samples at various ratios at an orally relevant shear rate was compared (FIG. 16).

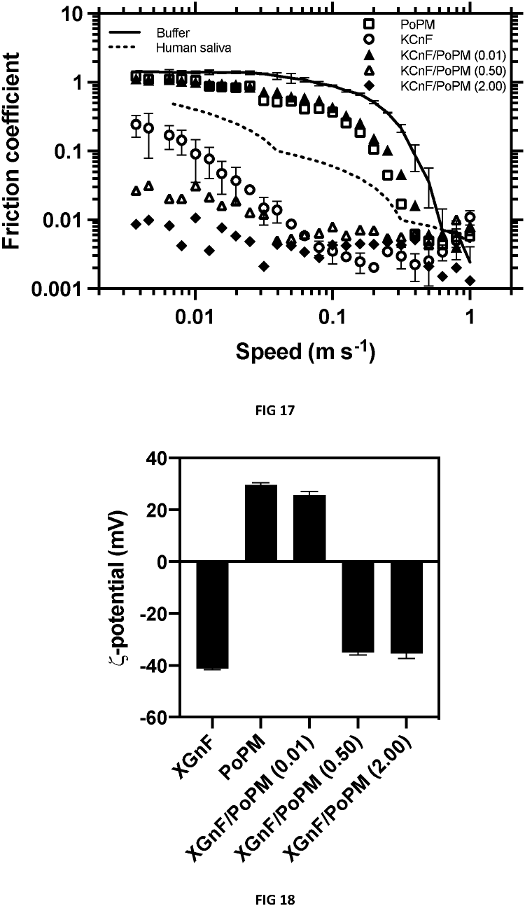

FIG. 17 shows the lubrication performance of the colloidosomes under orally relevant conditions, represented by the friction coefficient as a function of speeds. The lubrication properties of buffer and real human saliva are also shown for comparison purposes.

### Example 10: Analysis of Potato Protein Microgels Coated by Xanthan Gum Nanofibrils Manufactured in Example 5

The ζ-potential decreases upon increasing the concentration of XGnF (negatively charged) relatively to the concentration of PoPM (positively. charged) (FIG. 18). Raising the ratio, i.e., increasing the concentration of XGnF, allows the transition of the ζ-potential from positive to negative. In other words, upon increasing XGnF/PoPM ratio, PoPM particles become gradually negatively charged due to the gradual coverage by XGnF.

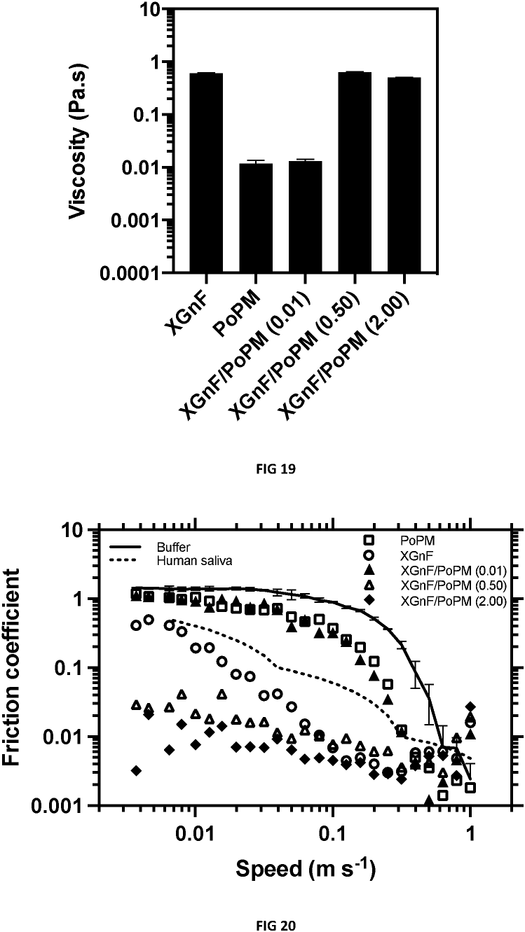

To evaluate the fluidity of the colloidosomes under relevant oral conditions, the apparent viscosity of our samples at various ratios at an orally relevant shear rate was compared (FIG. 19).

FIG. 20 shows the lubrication performance of the colloidosomes under orally relevant conditions, represented by the friction coefficient as a function of speeds. The lubrication of buffer and real human saliva are also shown for comparison purposes.

### Example 11: Surface Coverage

The ζ-potential of the colloidosomes of different formulations was measured. From those measurements, the % outer surface coverage can be calculated by the following equation:

$$\frac{3 \text{c}}{{\text{c}}_{s a t}} = - l n \left(\frac{{ζ}_{c} - {ζ}_{s a t}}{{ζ}_{0} - {ζ}_{s a t}}\right)$$

The following table illustrates the % outer surface coverage in colloidosomes of different formulations:

| | | | | | | | | | | |
|---|---|---|---|---|---|---|---|---|---|---|
| Ratio of KCnF/LFM (w/w) | 0.01 | 0.03 | 0.04 | 0.06 | 0.07 | 0.09 | 0.11 | 0.6 | 1.0 | 3.0 |
| %outer surface coverage (C/Cₛₐₜ) | 0.3 | 0.5 | 0.7 | 1.2 | 37.8 | 42.4 | 53 | 84.1 | 86.1 | 99.0 |

1. A formulation comprising:
(i) a proteinaceous microgel; and
(ii) one or more biopolymeric nanofibrils;
wherein either one of: (i) the proteinaceous microgel; and (ii) the one or more biopolymeric nanofibrils is positively charged, and the other is negatively charged;
wherein the one or more biopolymeric nanofibrils are associated with an outer surface of the oppositely charged proteinaceous or non-proteinaceous microgel; and
wherein the % outer surface coverage of the microgel by the nanofibrils is from about 50% to about 99%.

2. The formulation of claim 1, wherein the proteinaceous microgel is positively charged and the one or more biopolymeric nanofibrils are negatively charged.

3. The formulation of any preceding claim, wherein the weight ratio of one or more biopolymeric nanofibrils to proteinaceous microgel is from about 0.1:1 to about 10:1.

4. The formulation of claim 3, wherein the weight ratio of nanofibrils to microgel in the colloidosome is from about 0.2:1 to about 3:1.

5. The formulation of any preceding claim, wherein the proteinaceous microgel is selected from the group consisting of: lactoferrin, lysozyme, gelatin, milk protein, bovine serum albumin, whey protein, casein, caseinate, egg protein, albumin, gluten, gelatin Type B, pea protein, rice protein, legumin, corn protein, peanut protein and potato protein.

6. The formulation of claim 5, wherein the proteinaceous microgel is a lactoferrin microgel.

7. The formulation of any preceding claim, wherein the one or more biopolymeric nanofibrils are polysaccharide-based nanofibrils.

8. The formulation of claim 7, wherein the one or more biopolymeric nanofibrils are selected from the group consisting of: κ-carrageenan, i-carrageenan, λ-carrageenan, agar, agarose, alginate, pectin, dextran sulphate, cellulose, xanthan gum, gellan gum and any negatively-charged polysaccharide.

9. The formulation of claim 8, wherein the one or more biopolymeric nanofibrils are κ-carrageenan nanofibrils.

10. The formulation of any preceding claim, wherein the one or more biopolymeric nanofibrils are associated with an outer surface of the proteinaceous microgel by an electrostatic interaction.

11. The formulation of any preceding claim, wherein the one or more biopolymeric nanofibrils associated with the outer surface of the proteinaceous microgel result in an outer surface that has an overall negative charge.

12. The formulation of any preceding claim, wherein the formulation is a colloidosome.

13. The formulation of claim 12, wherein the colloidosome is no more than 1000 nm in diameter.

14. The formulation of any preceding claim, wherein the % outer surface coverage of the microgel by the nanofibrils (^{c}/cₛₐₜ) is calculated by the following equation:
$$\frac{3 \text{c}}{{\text{c}}_{s a t}} = - l n \left(\frac{{ζ}_{c} - {ζ}_{s a t}}{{ζ}_{0} - {ζ}_{s a t}}\right)$$
wherein:
ζₛₐₜ is the ζ-potential when the microgels are saturated with biopolymeric nanofibrils;
ζ₀ is the ζ-potential of the proteinaceous microgel in absence of the biopolymeric nanofibrils;
ζ_{c} is the ζ-potential of the formulation at biopolymeric nanofibril concentration c; and
cₛₐₜ is the minimum amount of the biopolymeric nanofibrils required to completely cover the surface of the proteinaceous microgel.

15. The formulation of any preceding claim, further comprising a pharmaceutically acceptable excipient.

16. The formulation of claim 15, wherein the pharmaceutically acceptable excipient comprises a buffered solution having a pH of from about 3.0 to about 4.0, or of about 7.0.

17. A method for preparing a formulation of any of claims 1 to 18, the method comprising:
(a) dissolving a proteinaceous material in a buffer solution and heating the resulting solution to form a proteinaceous microgel or a heat-set gel;
(b) when step (a) results in a heat-set gel, mixing the heat-set gel with the buffer solution and homogenising to form a proteinaceous microgel;
(c) adding the proteinaceous microgel of step (a) or step (b) to a solution of one or more biopolymeric nanofibrils to form the formulation,
wherein either one of: (i) the proteinaceous microgel; and (ii) the one or more biopolymeric nanofibrils is positively charged, and the other is negatively charged;
wherein the resulting formulation has the one or more biopolymeric nanofibrils associated with an outer surface of the proteinaceous microgel; and
wherein the amount of microgel that is added to nanofibrils is selected such that the % outer surface coverage of the microgel by the nanofibrils is from about 50% to about 99%.

18. A formulation obtainable or obtained by the method of claim 17.

19. The formulation of any of claims 1 to 16 for use as a medicament.

20. A use of a formulation of any of claims 1 to 16 as a lubricant food additive.

21. The formulation of any of claims 1 to 16 for use in the treatment of a disease or condition selected from or associated with: dry mouth, salivary gland diseases and disorders, chronic inflammatory autoimmune diseases, Sjögren’s syndrome, xerostomia, endocrine diseases, dysphagia, diabetes, neurologic diseases and disorders, psychogenic diseases, anxiety, nervousness, aging, HIV/AIDS and polypharmacy.

22. A method for the treatment of a disease or condition selected from or associated with: dry mouth, salivary gland diseases and disorders, chronic inflammatory autoimmune diseases, Sjögren’s syndrome, xerostomia, endocrine diseases, dysphagia, diabetes, neurologic diseases and disorders, psychogenic diseases, anxiety, nervousness, aging, HIV/AIDS and polypharmacy, wherein the method comprises administering a formulation of any of claims 1 to 16 to a patient in need thereof.
